(19) United States
(12) Patent Application Publication
(10) Pub. No.: US 2018/0036469 A1
(43) Pub. Date: Feb. 8, 2018

(54) Remote User Interfaces for Dialysis Systems

(71) Applicant: Fresenius Medical Care Holdings, Inc., Waltham, MA (US)

(72) Inventors: Martin Joseph Crnkovich, Walnut Creek, CA (US); David Yuds, Antioch, CA (US)

(21) Appl. No.: 15/229,435

(22) Filed: Aug. 5, 2016

(51) Int. Cl. A61M 1/26; H04B 1/3827; G06F 3/0346; H04L 29/08; G06F 3/0482; A61M 1/28; H04W 4/00

(52) U.S. Cl. CPC A61M 1/267; A61M 1/28; H04B 1/3833; H04W 4/008; H04L 67/12; G06F 3/0482; G06F 3/0346; A61M 2205/3584; A61M 2205/50; A61M 2205/502; A61M 2205/18; A61M 2205/52; A61M 2205/3592; H04W 84/12

(57) ABSTRACT

Mobile electronic devices can be used as remote user interfaces for medical devices such as dialysis machines. For example, this disclosure describes various ways mobile electronic devices can be networked with medical devices, and various ways users can remotely control the medical devices via the mobile electronic devices.

### TECHNICAL FIELD

This invention relates to remote user interfaces for one or more dialysis machines.

### BACKGROUND

Renal dysfunction or failure and, in particular, end-stage renal disease, causes the body to lose the ability to remove water and minerals, maintain acid-base balance, and control electrolyte and mineral concentrations within physiological ranges. Toxic uremic waste metabolites, including urea, creatinine, and uric acid, accumulate in the body's tissues which can result in a person's death if the filtration function of the kidney is not replaced.

Dialysis is commonly used to replace kidney function by removing these waste toxins and excess water. In one type of dialysis treatment—hemodialysis (HD)—toxins are filtered from a patient's blood externally in a hemodialysis machine. Blood passes from the patient through a dialyzer separated by a semi-permeable membrane from a large volume of externally-supplied dialysis solution. The waste and toxins dialyze out of the blood through the semi-permeable membrane into the dialysis solution, which is then typically discarded.

The dialysis solutions or dialysates used during hemodialysis typically contain sodium chloride and other electrolytes, such as calcium chloride or potassium chloride, a buffer substance, such as bicarbonate or acetate, and acid to establish a physiological pH, plus, optionally, glucose or another osmotic agent.

Another type of dialysis treatment is peritoneal dialysis (PD) that utilizes the patient's own peritoneum, a membranous lining of the abdominal body cavity. With its good perfusion properties, the peritoneum is capable of acting as a natural semi-permeable membrane for transferring water and waste products to a type of dialysate solution known as PD solution introduced temporarily into the patient's abdominal cavity. An access port is implanted in the patient's abdomen and the PD solution is infused usually by a pump into the patient's abdomen through a patient line and left to dwell for a period of time and then drained out. This procedure is usually repeated multiple times for a complete treatment.

Dialysis machines are typically equipped with user interfaces for receiving inputs and providing information to users.

### SUMMARY

Dialysis machines can be configured to communicate with a mobile electronic device that is adapted to be used as a remote user interface for the dialysis machines. Accordingly, a user can control the dialysis machines via the mobile electronic device.

In one aspect, the disclosure is directed to a dialysis machine that includes one or more processing units configured to transmit control data, a pump configured to pump medical fluid to and from a patient based at least in part on control data received from the one or more processing units, and a wireless communications interface configured to receive data from a mobile electronic device using a wireless communications protocol. The one or more processing units are configured to process input received from the wireless communications interface, and determine the control data based on the processed input.

Such a dialysis machine may optionally include one or more of the following features. The one or more processing units may be configured to cause a transmission, using the wireless communications interface, of user interface data to the mobile electronic device that enables the mobile electronic device to function as a remote user interface for the dialysis machine. The user interface data may enable the mobile electronic device to display, on a hardware display of the mobile electronic device, one or more selectable elements that correspond to respective selectable elements on a user interface display of the dialysis machine. The dialysis one or more selectable elements may include a selectable element for stopping the pump. The one or more selectable elements may include a selectable element for muting an alarm of the dialysis machine. The user interface data may enable the mobile electronic device to display, on a hardware display of the mobile electronic device, one or more parameters representing a current state of the dialysis machine. The data received from the mobile electronic device may include data indicative of one or more particular positions on a user interface display of the dialysis machine. The one or more processing units may be configured to cause a cursor or pointer to be displayed on the user interface display of the dialysis machine at the one or more particular positions in response to receiving the data indicative of the one or more particular positions. The data received from the mobile electronic device may include data indicative of a selection of a selectable element located at a particular position of the one or more particular positions. The one or more processing units may be configured to cause a selection of the selectable element in response to receiving the data indicative of a selection of a selectable element.

In another aspect, the disclosure is directed to a computer readable medium that stores computer executable instructions that, when executed by a hardware processor of a mobile electronic device, carry out operations including: (a) receiving, from a dialysis machine, data representing a current state of the dialysis machine; (b) displaying, on a hardware display of the mobile electronic device, a user interface that enables control of the dialysis machine, based on the data representing a current state of the dialysis machine; (c) receiving, at the user interface, user input representing a command to be executed by the dialysis machine; and (d) transmitting, using a wireless communication protocol, data representing the command to be executed by the dialysis machine.

Such a computer readable medium may optionally include one or more of the following features in some embodiments. The user input may represent a command to de-activate an alarm of the dialysis machine. The user input may represent a command to stop a pump of the dialysis machine. The user interface may include one or more selectable elements that correspond to respective selectable elements on a user interface display of the dialysis machine. The user interface may include one or more parameters representing the current state of the dialysis machine. The operations may include: (e) receiving, at the user interface, user input representing movement of a pointer or cursor location on the user interface; and (f) transmitting, using the wireless communication protocol, data representing the movement to be executed by the dialysis machine. The operations may include one or more operations that facilitate pairing the mobile electronic device and the dialysis machine.

In another aspect, this disclosure is directed to a mobile electronic device including a hardware processor, a wireless communications interface configured to transmit data using a wireless communications protocol, and a movement sensor. The hardware processor is configured to receive data from the movement sensor representing movement of the mobile electronic device and, based on the received data, use the wireless communications interface to transmit, to a dialysis machine, data usable by the dialysis machine to affect a position of a cursor on a user interface of the dialysis machine.

Such a mobile electronic device may optionally include one or more of the following features. The hardware processor may be configured to facilitate pairing of the mobile electronic device and the dialysis machine such that wireless communications can occur using the wireless communications protocol. The movement sensor may include at least one of a gyroscope or an accelerometer. The mobile electronic device may include a hardware display coupled to the hardware processor. The hardware processor may be configured to receive data representing a user input made to the hardware display and to transmit, to the dialysis machine, data usable by the dialysis machine for selecting a selectable element displayed on the user interface of the dialysis machine.

In another aspect, this disclosure is directed to a dialysis machine including one or more processing units configured to transmit control data, a pump configured to pump medical fluid to and from a patient based at least in part on control data received from the one or more processing units, and a wireless communications interface configured to receive data from a mobile electronic device using a wireless communications protocol. The data received from the mobile electronic device includes data indicative of one or more particular positions on a user interface display of the dialysis machine. The one or more processing units are configured to cause a cursor or pointer to be displayed on the user interface display of the dialysis machine at the one or more particular positions in response to receiving the data indicative of the one or more particular positions.

Such a dialysis machine may optionally include one or more of the following features. The data received from the mobile electronic device may include data indicative of a selection of a selectable element located at a particular position of the one or more particular positions. The one or more processing units may be configured to cause a selection of the selectable element in response to receiving the data indicative of a selection of a selectable element.

In another aspect, this disclosure is directed to a dialysis system including a first dialysis machine, a second dialysis machine, and a mobile electronic device. The first dialysis machine includes one or more first processing units configured to transmit control data, a first pump configured to pump medical fluid to and from a first patient based at least in part on first control data received from the first one or more processing units, and a first wireless communications interface configured to receive data from a mobile electronic device using a wireless communications protocol. The first one or more processing units are configured to process input received from the first wireless communications interface and to determine the first control data based on the processed input. The second dialysis machine includes one or more second processing units configured to transmit control data, a second pump configured to pump medical fluid to and from a second patient based at least in part on control data received from the one or more processing units, and a second wireless communications interface configured to receive data from a mobile electronic device using a wireless communications protocol. The second one or more processing units are configured to process input received from the second wireless communications interface and to determine the second control data based on the processed input. The mobile electronic device includes a hardware processor and a third wireless communications interface configured to transmit data to the first and second dialysis machines using the wireless communications protocol.

Such a dialysis system may optionally include one or more of the following features. At least one of the first and second dialysis machines may comprise a hemodialysis machine. At least one of the first and second dialysis machines may comprise a peritoneal dialysis machine. The mobile electronic device may be configured to transmit data associated with a first user interface of the first dialysis machine and configured to transmit data associated with a second user interface of the second dialysis machine.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A shows a communications network that includes multiple hemodialysis machines and a mobile electronic device configured to be used as a remote user interface for the hemodialysis machines.

FIG. 1B shows a communications network that includes multiple peritoneal dialysis (PD) machines and a mobile electronic device configured to be used as a remote user interface for the PD machines.

FIG. 2 shows an example configuration of a mobile electronic device display that can be used as a remote user interface for a hemodialysis machine.

FIG. 3 shows another example configuration of a mobile electronic device display that can be used as a remote user interface for a hemodialysis machine.

FIG. 4 shows another example configuration of a mobile electronic device display that can be used as a remote user interface for a hemodialysis machine.

FIG. 5 shows simulated patient identification data displayed on a mobile electronic device. Such patient identification data can be communicated using the mobile electronic device as a remote user interface for a hemodialysis machine.

FIG. 6 shows a communications network that includes multiple hemodialysis machines and a mobile electronic device configured to be used as a remote user interface for muting alarms of the hemodialysis machines.

FIG. 7 shows a mobile electronic device being used as a remote user interface to control a cursor position on display of a hemodialysis machine.

FIG. 8 shows a single hemodialysis machine in one-to-one communication with a mobile electronic device that is being used as a remote user interface for the single hemodialysis machine.

FIG. 9 shows an example of a processing system of a hemodialysis machine.

Like reference symbols in the various drawings indicate like elements.

### DETAILED DESCRIPTION

This disclosure describes how mobile electronic devices can be used as remote user interfaces for medical devices such as dialysis machines. For example, this disclosure describes various ways mobile electronic devices can be networked with medical devices, and various ways users can remotely control the medical devices via the mobile electronic devices.

Medical devices (e.g., dialysis machines, dialysis machine components, dialysis machine accessories, etc.) can be configured to communicate with other devices through a connection between the devices. A “connection” established between devices as described herein refers to electronic communication between two or more devices such that data can be communicated between the devices. The connection can be a unidirectional connection (in which data travels one way) or a bidirectional connection (in which data travels both ways). The connection can be hard-wired, wireless, or a combination of both.

In addition to the medical devices themselves, such a system can include one or more other electronic devices that are configured to remotely control the medical devices. For example, in some cases mobile electronic devices (e.g., smart phones, tablet computers, smart watches, PDAs, wearable computers, and the like) can be configured for use as a remote user interface for the medical devices. A user can manipulate such a mobile electronic device to, for example, enter commands that are transmitted to a medical device to control the medical device.

Various types of wireless communication technologies and protocols can be used in such a system of medical devices that are configured for communications. For example, without limitation, wireless technologies such as Bluetooth™, WiFi, radio-frequency identification (RFID), ANT+, near field communication (NFC), infrared (IR), and other such technologies can be utilized. The systems described herein may use appropriate encryption and security standards and protocols in connection with the transmission of sensitive and/or protected data in accordance with statutory and regulatory requirements.

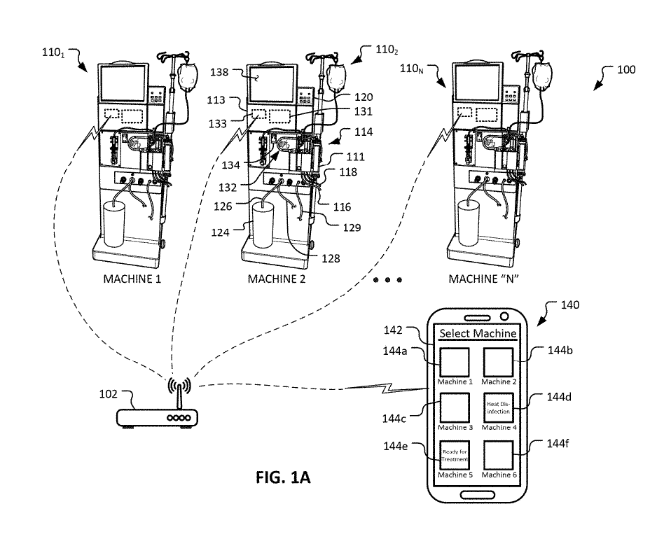

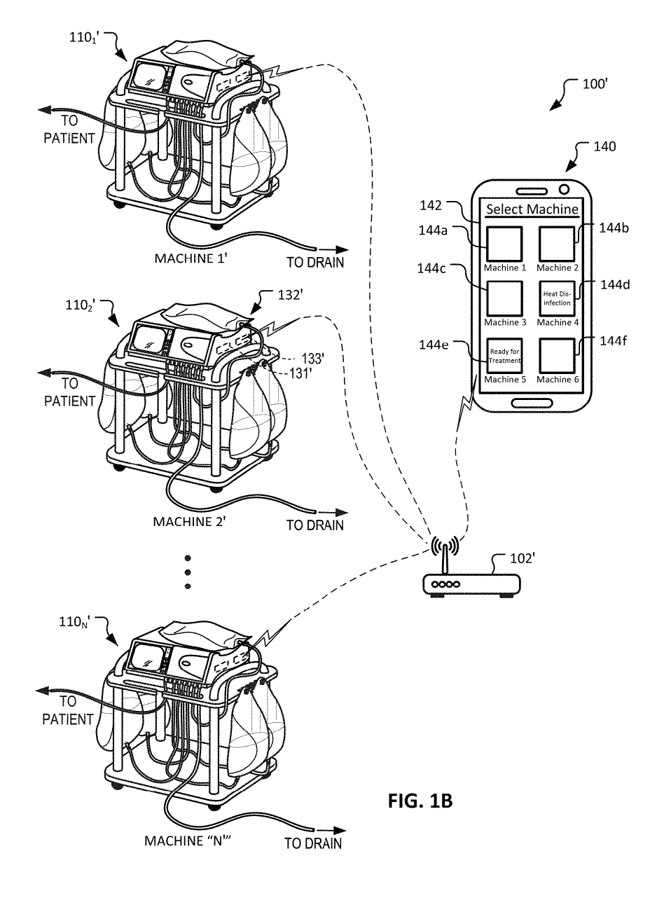

With reference to FIG. 1A, an example medical device system 100 can include multiple hemodialysis machines 110₁, 110₂ . . . 110_{N} that are configured to securely communicate with a mobile electronic device 140 adapted to be used as a remote user interface for the hemodialysis machines 110₁, 110₂ . . . 110_{N}. In some cases, the system 100 may be used in the context of a hospital, clinic, or kidney dialysis center, for example, and communication may be facilitated through a wireless router or gateway 102 and/or other network device that establishes a secure connection between the hemodialysis machines 110₁, 110₂ . . . 110_{N} and the mobile electronic device 140. Although the system 100 is described as including the multiple hemodialysis machines 110₁, 110₂ . . . 110_{N} by way of example, it is explicitly noted that the inventive concepts may be used in connection with other types of medical devices and treatments including, but not limited to, peritoneal dialysis (PD) systems (see, e.g., FIG. 1B).

In this example, the wireless gateway 102 wirelessly receives and transmits communications of the system 100 using WiFi. Alternatively or additionally, any and all other types of wired and wireless communications can be used for the system 100. The security of the communications of the system 100 can be controlled using secure login access techniques. The system 100 may also include one or more other devices and/or systems such as, but not limited to, medical information systems, databases, servers, internet portals, computer workstations, and the like. The hemodialysis machines 110₁, 110₂ . . . 110_{N} are used to treat patients whose kidneys are not functioning properly. Any number of the hemodialysis machines 110₁, 110₂ . . . 110_{N} can be included in the system 100. The system described herein may also be used for dialysis treatments in connection with types of medical devices other than hemodialysis machines, such as PD treatments performed using PD machines.

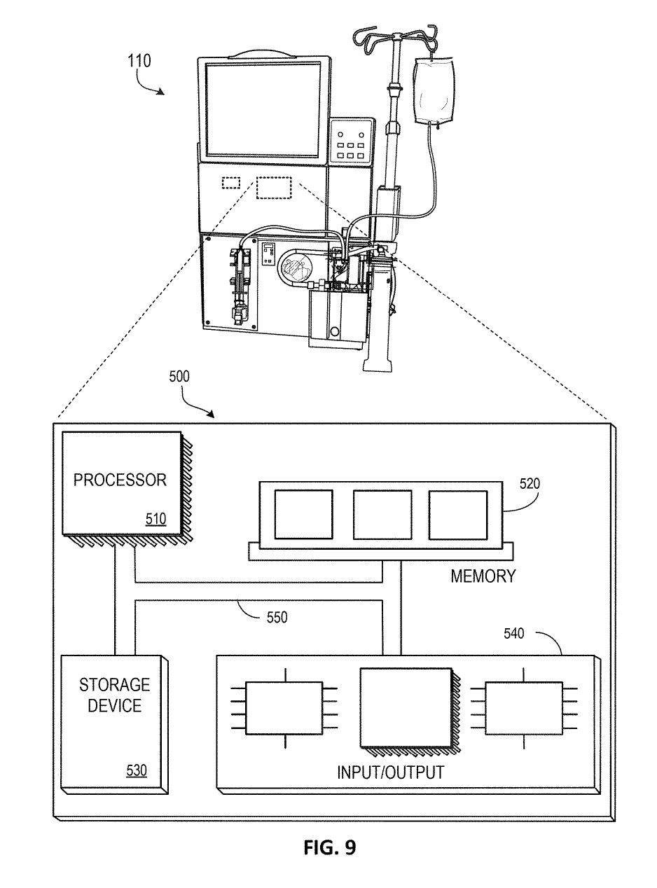

For example, FIG. 1B is an illustration of a system 100′ including multiple PD machines 110₁′, 110₂′ . . . 110_{N}′ that are configured to securely communicate with the mobile electronic device 140 adapted to be used as a remote user interface for one or more of the PD machines 110₁′, 110₂′ . . . 110_{N}′. As exemplified by the PD machine 110₂′, the PD machines 110₁′, 110₂′ . . . 110_{N}′ each include a blood pump 132′, one or more processing units 131′ (described further in reference to FIG. 9), and a wireless communications interface 133′ (a WiFi transceiver in this example). In some cases, the system 100′ may be used in the context of a home dialysis setting in which communication may be facilitated between the remotely located PD machines 110₁′, 110₂′ . . . 110_{N}′ at one or more homes and the mobile electronic device 140 via a gateway 102′. In some cases, the gateway 102′ may represent multiple gateway devices, or components thereof, that are separately located at the remote locations, such as in the one or more homes, and corresponding to each of the one or more remotely located PD machines 110₁′, 110₂′ . . . 110_{N}′.

Referring back to FIG. 1A, as an illustrative example, the hemodialysis machine 110₂ will be used to briefly describe the hemodialysis machines 110₁, 110₂ . . . 110_{N} in further detail. It should be understood that the other hemodialysis machines 110₁ . . . 110_{N} can be analogous to the hemodialysis machine 110₂.

The hemodialysis machine 110₂ is connected to a disposable blood component set 114 that partially forms a blood circuit. During hemodialysis treatment, an operator connects arterial and venous patient lines 116, 118 of the blood component set 114 to a patient to complete the blood circuit.

The blood component set 114 is secured to the front of the hemodialysis machine 110₂. A blood pump 132 is used to circulate blood through the blood circuit. The hemodialysis machine 110₂ can also include various other instruments capable of monitoring and/or controlling the blood flowing through the blood circuit.

The operator of the hemodialysis machine 110₂ can use a blood pump control interface 134 to operate the blood pump 132. In some embodiments, the blood pump module 134 includes components such as a display window, a start/stop key, an up key, a down key, a level adjust key, and an arterial pressure port. The display window displays the blood flow rate setting during blood pump operation. The start/stop key starts and stops the blood pump 132. The up and down keys increase and decrease the speed of the blood pump 132. The level adjust key raises a level of fluid in an arterial drip chamber.

The hemodialysis machine 110₂ further includes a dialysate circuit formed by the dialyzer 111, various other dialysate components, and dialysate lines connected to the hemodialysis machine 110₂. Many of these dialysate components and dialysate lines are inside the housing 113 of the hemodialysis machine 110₂ and are thus not visible in FIG. 1A. During treatment, while the blood pump 132 circulates medical fluid such as blood through the blood circuit, dialysate pumps (not shown) circulate medical fluid such as dialysate through the dialysate circuit.

A dialysate container 124 is connected to the hemodialysis machine 110₂ via a dialysate supply line 126. A drain line 128 and an ultrafiltration line 129 also extend from the hemodialysis machine 110₂. The dialysate supply line 126, the drain line 128, and the ultrafiltration line 129 are fluidly connected to the various dialysate components and dialysate lines inside the housing 103 of the hemodialysis machine 110₂ that form part of the dialysate circuit. During hemodialysis, the dialysate supply line 126 carries fresh dialysate from the dialysate container 124 to the portion of the dialysate circuit located inside the hemodialysis machine 110₂. As noted above, the fresh dialysate is circulated through various dialysate lines and dialysate components, including the dialyzer 110, that form the dialysate circuit. As the dialysate passes through the dialyzer 110, it collects toxins from the patient's blood. The resulting spent dialysate is carried from the dialysate circuit to a drain via the drain line 128. When ultrafiltration is performed during treatment, a combination of spent dialysate (described below) and excess fluid drawn from the patient is carried to the drain via the ultrafiltration line 129.

The dialyzer 110 serves as a filter for the patient's blood. The dialysate passes through the dialyzer 110 along with the blood, as described above. A semi-permeable structure (e.g., a semi-permeable membrane and/or semi-permeable microtubes) within the dialyzer 110 separates blood and dialysate passing through the dialyzer 110. This arrangement allows the dialysate to collect toxins from the patient's blood. The filtered blood exiting the dialyzer 110 is returned to the patient. The dialysate exiting the dialyzer 110 includes toxins removed from the blood and is commonly referred to as “spent dialysate.” The spent dialysate is routed from the dialyzer 110 to a drain.

The hemodialysis machine 110₂ includes a user interface with input/output devices such as a touch screen 138, a control panel 120, and the blood pump control interface 134. The touch screen 138 and the control panel 120 allow the operator to input various different treatment parameters to the hemodialysis machine 110₂ and to otherwise control the hemodialysis machine 110₂. The touch screen 138 displays information to the operator of the hemodialysis system 110₂.

The hemodialysis machine 110₂ also includes one or more processing units 131 (described further in reference to FIG. 9) and a wireless communications interface 133 (a WiFi transceiver in this example). The processing units 131 are configured to control hemodialysis machine 110₂. For example, among other things, the processing units 131 are configured to determine and transmit control data for controlling the blood pump 132. Such control data may include, but is not limited to, electronic signals that facilitate starting and stopping of the blood pump 132, controlling the speed of the blood pump 132, controlling the acceleration and deceleration of the blood pump 132, and the like.

In the depicted embodiment, the processing units 131 are also configured to communicate (send and receive) data via the wireless communications interface 133. In this manner, for example, the hemodialysis machine 110₂ is configured to communicate with the mobile electronic device 140 such that the mobile electronic device 140 can be used as a remote user interface for the hemodialysis machine 110₂. Accordingly, data from the hemodialysis machine 110₂ can be displayed by the mobile electronic device 140, and commands for controlling the hemodialysis machine 110₂ can be entered into the mobile electronic device 140 and transmitted to the processing units 131 via the wireless communications interface 133. The processing units 131 are configured to receive and process input from the wireless communications interface 133 and to determine control data (e.g., for controlling the blood pump 132) based on the processed input.

The example medical device system 100 also includes the mobile electronic device 140. The mobile electronic device 140 is adapted to be used as a remote user interface for the hemodialysis machines 110₁, 110₂ . . . 110_{N}. In the depicted example, the mobile electronic device 140 is a smart phone. Alternatively, in some embodiments the mobile electronic device 140 can be another type of mobile computing device such as, but not limited to, a tablet computer, laptop computer, a smart watch and other types of wearable computers, a PDA, and the like. In some cases, two or more mobile electronic devices 140 can be used in the same medical device system 100.

In the depicted example, the mobile electronic device 140 is running a computer program of executable instructions. In some cases, the executable instructions can be downloaded to the mobile electronic device 140 and saved in its memory. In some cases, the executable instructions, or portions thereof, can be stored on another computer system in communication with the mobile electronic device 140 such that the mobile electronic device operates as described herein.

The executable instructions configure the mobile electronic device 140 to carry out multiple functions within the context of the medical device system 100. For example, the mobile device 140 can receive data from the hemodialysis machines 110₁, 110₂ . . . 110_{N} that represents a current state of the hemodialysis machines 110₁, 110₂ . . . 110_{N}. The mobile device 140 can also receive user input for controlling the hemodialysis machines 110₁, 110₂ . . . 110_{N}. The user input can be sent from the mobile electronic device 140, using the router 102, and received by the hemodialysis machines 110₁, 110₂ . . . 110_{N}. The hemodialysis machines 110₁, 110₂ . . . 110_{N} can then perform the actions that correspond to the user input. Hence, the mobile electronic device 140 operates as a remote user interface for the hemodialysis machines 110₁, 110₂ . . . 110_{N}.

The executable instructions also configure the mobile electronic device 140 to be able to display multiple types of user interface information on a hardware touchscreen display 142 of the mobile device 140. In the depicted example, the hardware touchscreen display 142 is displaying selectable elements 144a, 144b, 144c, 144d, 144e, 144f that represent and correspond to individual hemodialysis machines of the hemodialysis machines 110₁, 110₂ . . . 110_{N}. Accordingly, a user can selectively activate (e.g., tap, double tap, touch for at least a threshold period of time, etc.) one of the selectable elements 144a, 144b, 144c, 144d, 144e, 144f when the user desires to use the mobile electronic device 140 to interface with a particular one of the hemodialysis machines 110₁, 110₂ . . . 110_{N}.

While six selectable elements 144a, 144b, 144c, 144d, 144e, 144f that represent and correspond to six individual hemodialysis machines are depicted, any number of selectable elements and corresponding hemodialysis machines can be included. In some cases when a high number of selectable elements and corresponding hemodialysis machines are included in the medical device system 100, multiple screens can be used to display all the selectable elements. In some such cases, a user can simply “swipe” his/her finger across the hardware touchscreen display 142 to switch between the multiple screens.

The depicted example also illustrates that the status of the hemodialysis machines 110₁, 110₂ . . . 110_{N} can be displayed on the hardware touchscreen display 142 of the mobile electronic device 140. For example, the hardware touchscreen display 142 shows that the status associated with the selectable element 144d is “Heat Disinfection,” and the status associated with the selectable element 144e is “Ready for Treatment.” The display of such descriptions can provide a user of the mobile electronic device 140 with a convenient status overview pertaining to the multiple hemodialysis machines 110₁, 110₂ . . . 110_{N}. In addition to displaying information on the hardware touchscreen display 142, in some embodiments the mobile electronic device 140 can output information audibly and/or tactilely.

The executable instructions also configure the mobile electronic device 140 to be able to receive user input. As described above, in the depicted example user input can be received via the hardware touchscreen display 142 of the mobile electronic device 140. That is, the mobile electronic device 140 can receive various types of touch inputs (e.g., tap, swipe, drag, gestures, multi-touch gestures, text input, soft key inputs, stylus inputs, etc.).

The executable instructions can also configure the mobile electronic device 140 to be able to receive other types of user input. In some embodiments, the mobile electronic device 140 can be configured to receive user input in the form of voice commands. In some embodiments, the mobile electronic device 140 can be configured to receive user input in forms such as, but not limited to, tilting, moving, orienting, and posing the mobile electronic device 140 in predefined manners that are associated with particular types of user inputs. In some embodiments, one or more movement sensors within the mobile electronic device 140 can be used to detect such types of user inputs. In response, the movement sensors can generate data representative of the motion and/or orientation of the mobile electronic device 140. For example, in some cases movement sensors such as accelerometers and/or gyroscopic sensors within the mobile electronic device 140 may be utilized for such types of user inputs. In some embodiments, the mobile electronic device 140 can be configured to receive user input using one or more buttons or switches coupled to the mobile electronic device 140.

The executable instructions can also configure the mobile electronic device 140 to be able to transmit data representing one or more commands for operating the hemodialysis machines 110₁, 110₂ . . . 110_{N}. The transmission can be a wireless transmission using various types of wireless technologies and protocols such as, but not limited to, Bluetooth™, WiFi, RFID, ANT+, NFC, IR, and other such technologies. As described further herein, multiple types of commands for operating the hemodialysis machines 110₁, 110₂ . . . 110_{N} can be transmitted from the mobile electronic device 140 to the hemodialysis machines 110₁, 110₂ . . . 110_{N}. Such commands can include, but are not limited to, deactivation of alarms, starting or stopping a pump, pausing a pump, starting or stopping a treatment procedure, setting operational parameters, adjusting operational parameters, downloading patient information, and the like. The commands may also include downloading a prescription for the dialysis treatment of a patient in which the prescription is prepared by a doctor and/or appropriate clinician and is transmitted to one or more of the hemodialysis machine 110₁, 110₂ . . . 110_{N} using the mobile electronic device 140 and applying treatment parameters at the one or more of the hemodialysis machine 110₁, 110₂ . . . 110_{N} with respect to a dialysis treatment performed therewith. It is noted that the commands described herein may also be applied in connection with other types of medical devices, including PD machines (see, e.g., FIG. 1B).

Still referring to FIG. 1A, when the user of the mobile electronic device 140 desires to interface with a particular one of the hemodialysis machines 110₁, 110₂ . . . 110_{N}, a selection of one of the selectable elements 144a, 144b, 144c, 144d, 144e, 144f can be made. For example, when the user desires to interface with the hemodialysis machine 110₁, the user can enter an input that activates the selectable element 144a.

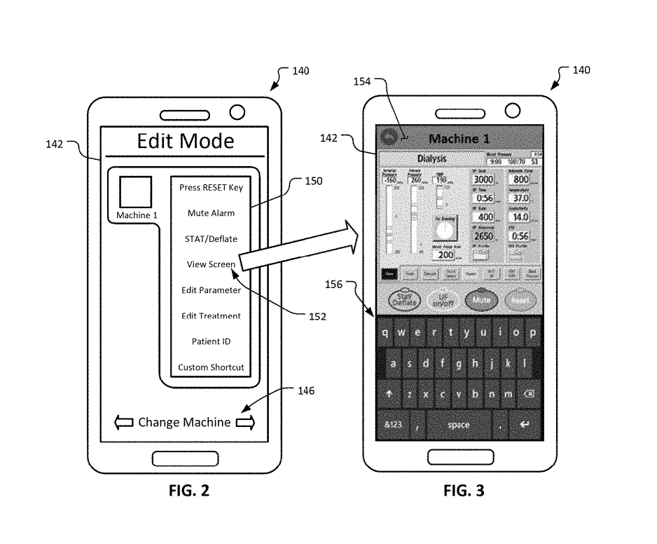

Referring now also to FIG. 2, in the depicted embodiment, activation of the selectable element 144a causes the generation and display on the hardware touchscreen display 142 of a menu of commands 150 particularly pertaining to the hemodialysis machine 110₁ (also referred to in the figures as “Machine 1”). Analogously, activation of any one of the other selectable elements 144b, 144c, 144d, 144e, 144f would result in the generation and display on the hardware touchscreen display 142 of a menu of commands particularly pertaining to a hemodialysis machine represented by the activated selectable element 144b, 144c, 144d, 144e, 144f Additionally, the menu of commands particularly pertaining to other hemodialysis machines can be accessed by a lateral finger-swiping input on the hardware touchscreen display 142 in a “Change Machine” field 146.

The menu of commands 150 shown merely includes non-limiting examples of the types of commands that can be presented to a user. In some cases, a selection of a command from the menu of commands 150 may cause a transmission from the mobile electronic device 140 of data representing the selected command to be executed by the corresponding hemodialysis machine. In some cases, a selection of a command from the menu of commands 150 may cause the generation and display on the hardware touchscreen display 142 of a sub-menu or other type of information pertaining to the command selected.

Referring also to FIG. 3, in the depicted simulated example the command “View Screen” 152 has been selected, resulting in the generation and display on the hardware touchscreen display 142 of a replication of a user interface display 154 of the hemodialysis machine 110₁ (“Machine 1”). As shown, the replicated user interface display 154 includes one or more numerically-represented and/or graphically-represented parameters associated with a current state of the hemodialysis machine 110₁. In addition, the replicated user interface display 154 includes data entry fields that the user can activate and then enter alphanumeric data into using soft keys 156 on the hardware touchscreen display 142 (or enter by voice input, for example).

The replicated user interface display 154 also includes one or more selectable elements for example commands such as, muting an alarm, resetting the machine, stopping the pump, and so on. The replicated user interface display 154 also includes one or more selectable elements (e.g., tabs, folders, etc.) for switching between various user interface screens of the hemodialysis machine 110₁. It should be understood that the replicated user interface display 154 can be used to display any of the information that would be displayed on the user interface display(s) of the hemodialysis machine 110₁. Additionally, all types of user input can be received via the replicated user interface display 154 that are receivable by the user interface(s) of the hemodialysis machine 110₁.

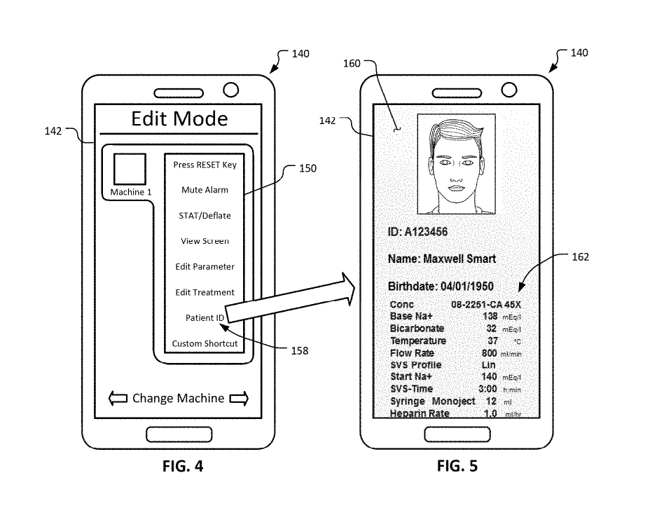

Referring to FIGS. 4 and 5, in another simulated example a command “Patient ID” 158 is selected from the menu of commands 150. In response to the selection of the command “Patient ID” 158, patient data 160 (which can include a hemodialysis prescription 162 in some embodiments) can be accessed and displayed on the hardware touchscreen display 142.

In some cases, the patient data 160 can be accessed from a medical information system that is in communication with the medical device system 100. In some such cases, the patient data 160 can be conveniently downloaded from the medical information system to the hemodialysis machine 110₁ using the command “Patient ID” 158. Additionally or alternatively, the patient data 160 can be accessed from the hemodialysis machine 110₁ using the command “Patient ID” 158 and thereby displayed on the hardware touchscreen display 142 for viewing by the user of the mobile electronic device 140.

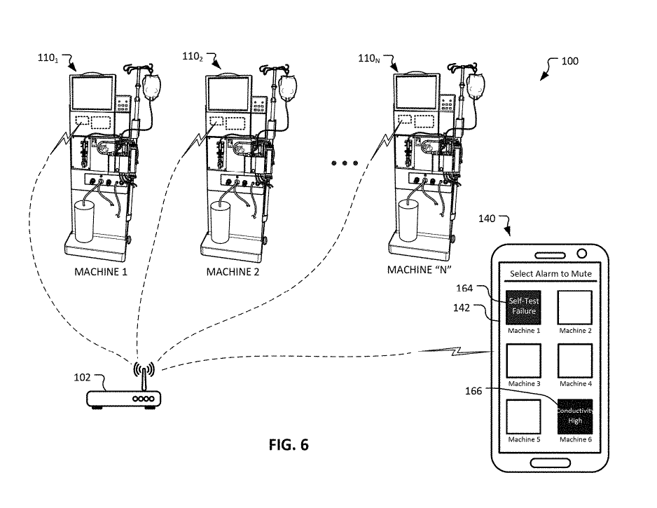

Referring to FIG. 6, the mobile electronic device 140 can also conveniently facilitate remote user notification and muting (e.g., acknowledgement, clearing, resetting, restarting, etc.) of alarms of the hemodialysis machines 110₁, 110₂ . . . 110_{N}. In addition to alarms, remote user notifications can similarly be provided for machine statuses such as, but not limited to, a treatment procedure has been completed, the machine needs intervention, and the like.

In the depicted example, the hardware touchscreen display 142 is notifying the user of a “Self-Test Failure” alarm 164 occurring at Machine 1, and a “Conductivity High” alarm 166 occurring at Machine 6. It should be understood that these are merely examples of the various types of alarms that can be used for the medical device system 100. Audible and/or tactile output may also be provided via the mobile electronic device 140 in the event of such alarms. If desired, the user can individually select either of the alarms 164 and 166 to obtain further information regarding the alarms 164 and 166, and/or to mute the alarms 164 and 166.

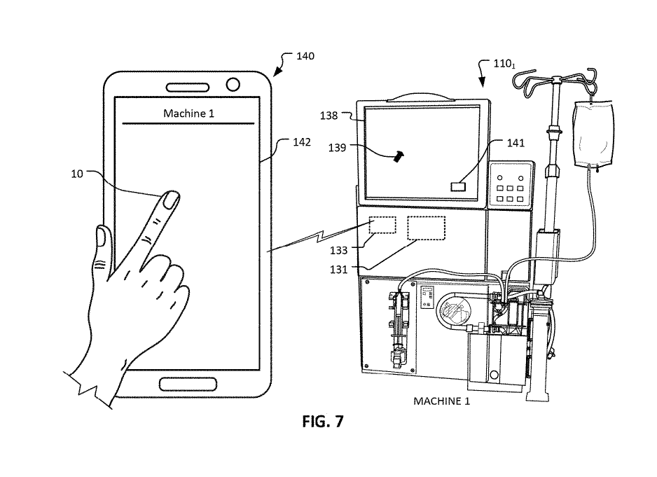

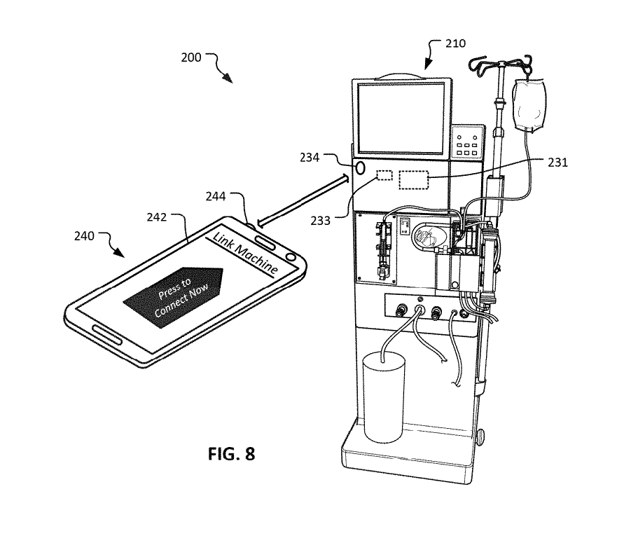

Referring to FIG. 7, in some embodiments the mobile electronic device 140 is configured to receive user input that facilitates remote user control of the position of a pointer (or cursor and the like) on a user interface display of a hemodialysis machine. This technique can also be used to make selections of selectable elements on the user interface display of the hemodialysis machine. In this mode of operation, the mobile electronic device 140 operates in a manner like a wireless touchpad for the hemodialysis machine. This mode of operation may be used in the context of the medical device system 100, and in the context of one-to-one communication between a mobile electronic device and a single hemodialysis machine as described below (in reference to FIG. 8).

In the depicted example, a user's finger 10 is touching the hardware touchscreen display 142 and thereby controlling the position of a pointer 139 on the touch screen 138 of the hemodialysis machine 110₁. As the user slides his/her finger 10 across the hardware touchscreen display 142, the pointer 139 moves correspondingly across the touch screen 138. When the pointer 139 is positioned over a selectable element displayed on the touch screen 138 (e.g., selectable element 141), the user can activate the selectable element 141 via the mobile electronic device 140. For example, the user can tap one or more times on the hardware touchscreen display 142 at the position of the pointer 139 while the pointer 139 is over the selectable element 141. Other selection techniques can also be used.

The mobile electronic device 140 can also be used to control the hemodialysis machine 110₁ in additional manners. For example, in some embodiments the user can swipe his/her finger 10 across the hardware touchscreen display 142 to change pages of information displayed on the touch screen 138 of the hemodialysis machine 110₁. In some embodiments, alphanumeric characters can be entered into data fields displayed on the touch screen 138 of the hemodialysis machine 110₁ using the finger 10 to trace the characters on the hardware touchscreen display 142. In some embodiments, a soft keyboard can be selectively displayed the hardware touchscreen display 142 and used to enter alphanumeric characters into data fields displayed on the touch screen 138 of the hemodialysis machine 110₁.

In some embodiments, by manipulating the three-dimensional spatial orientation of the mobile electronic device 140 the position of the pointer 139 can be remotely controlled. That is, in some embodiments the user can tilt, rotate, or otherwise move the mobile electronic device 140, and the position of the pointer 139 on the touch screen 138 will move correspondingly. Accelerometers and/or gyroscopic sensors within the mobile electronic device 140 may be utilized for such types of user inputs. For example, tilting the left edge of the mobile electronic device 140 downward may cause the pointer 139 to move leftward on the touch screen 138. Similarly, tilting the right edge of the mobile electronic device 140 downward may cause the pointer 139 to move rightward on the touch screen 138; tilting the top edge of the mobile electronic device 140 upward may cause the pointer 139 to move upward on the touch screen 138; and tilting the top edge of the mobile electronic device 140 downward may cause the pointer 139 to move downward on the touch screen 138. Such techniques can be used to position the pointer 139 at a desired location on the touch screen 138 of the hemodialysis machine 110₁ without actually touching the touch screen 138.

Referring to FIG. 8, a system 200 can use short-range wireless technology protocols for direct one-to-one communications between a mobile electronic device 240 and a single hemodialysis machine 210. Whereas, as described above, the medical device system 100 includes the multiple hemodialysis machines 110₁, 110₂ . . . 110_{N} that are networked with the mobile electronic device 140, the system 200 includes just one hemodialysis machine 210 at a time that is in communication with the mobile electronic device 240.

While the mobile electronic device 240 is in communication with the hemodialysis machine 210, the mobile electronic device 240 can be used as a remote user interface for the hemodialysis machine 210 in any of the manners described herein. The system 200 can use short-range wireless technology protocols such as, but not limited to, NFC, Bluetooth™, and IR.

In the depicted example, IR is being used for communications between the mobile electronic device 240 and the hemodialysis machine 210. The mobile electronic device 240 includes an IR transceiver 244, and the hemodialysis machine 210 includes a compatible IR transceiver 234. Data formatted as IR energy can be beamed between the mobile electronic device 240 and the hemodialysis machine 210 to provide for two-way communications so that mobile electronic device 240 can be used as a remote user interface for the hemodialysis machine 210.

In some embodiments, the hemodialysis machine 210 also includes a wireless communication interface 233 that is electrically coupled with the IR transceiver 234 and one or more processing units 231 of the hemodialysis machine 210. In some such embodiments, the wireless communication interface 233 can also facilitate other types of wireless communication (e.g., WiFi, etc.) such that the hemodialysis machine 210 can be remotely controlled using either the network approach of medical device system 100 or the one-to-one approach of system 200.

FIG. 9 is a block diagram of an example computer system 500. For example, the one or more processing units 131 of the hemodialysis machines described above could be an example of the system 500 described here. The system 500 includes a processor 510, a memory 520, a storage device 530, and an input/output device 540. Each of the components 510, 520, 530, and 540 can be interconnected, for example, using a system bus 550. The processor 510 is capable of processing instructions for execution within the system 500. The processor 510 can be a single-threaded processor, a multi-threaded processor, or a quantum computer. The processor 510 is capable of processing instructions stored in the memory 520 or on the storage device 530. The processor 510 may execute operations such as causing the dialysis system to carry out functions related to voice commands, voice alarms, and voice instructions.

The memory 520 stores information within the system 500. In some implementations, the memory 520 is a computer-readable medium. The memory 520 can, for example, be a volatile memory unit or a non-volatile memory unit. In some implementations, the memory 520 stores information related to patients' identities. The information related to patients' identities can include patient names, identification numbers, or values that correspond to patient names or identification numbers, among others.

The storage device 530 is capable of providing mass storage for the system 500. In some implementations, the storage device 530 is a non-transitory computer-readable medium. The storage device 530 can include, for example, a hard disk device, an optical disk device, a solid-date drive, a flash drive, magnetic tape, or some other large capacity storage device. The storage device 530 may alternatively be a cloud storage device, e.g., a logical storage device including multiple physical storage devices distributed on a network and accessed using a network. In some implementations, the information stored on the memory 520, such as the information related to patients' identities, can also or instead be stored on the storage device 530.

The input/output device 540 provides input/output operations for the system 500. In some implementations, the input/output device 540 includes one or more of network interface devices (e.g., an Ethernet card), a serial communication device (e.g., an RS-232 10 port), and/or a wireless interface device (e.g., a short-range wireless communication device, an 802.11 card, a 3G wireless modem, or a 4G wireless modem). In some implementations, the input/output device 540 includes driver devices configured to receive input data and send output data to other input/output devices, e.g., a short-range wireless communication device, a keyboard, a printer, other wireless communication modules (such as the wireless communications interface 133), and display devices (such as the touch screen display 138). In some implementations, mobile computing devices, mobile communication devices, and other devices are used.

In some implementations, the system 500 is a microcontroller. A microcontroller is a device that contains multiple elements of a computer system in a single electronics package. For example, the single electronics package could contain the processor 510, the memory 520, the storage device 530, and input/output devices 540.

Although an example processing system 500 has been described in FIG. 9, implementations of the subject matter and the functional operations described above can be implemented in other types of digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible program carrier, for example a computer-readable medium, for execution by, or to control the operation of, a processing system. The computer readable medium can be a machine readable storage device, a machine readable storage substrate, a memory device, a composition of matter effecting a machine readable propagated signal, or a combination of one or more of them.

The term “computer system” may encompass all apparatus, devices, and machines for processing data, including by way of example a programmable processor, a computer, or multiple processors or computers. A processing system can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, executable logic, or code) can be written in any form of programming language, including compiled or interpreted languages, or declarative or procedural languages, and it can be deployed in any form, including as a standalone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

Computer readable media suitable for storing computer program instructions and data include all forms of non-volatile or volatile memory, media and memory devices, including by way of example semiconductor memory devices, e.g., EPROM, EEPROM, and flash memory devices; magnetic disks, e.g., internal hard disks or removable disks or magnetic tapes; magneto optical disks; and CD-ROM and DVD-ROM disks. The processor and the memory can be supplemented by, or incorporated in, special purpose logic circuitry. The components of the system can be interconnected by any form or medium of digital data communication, e.g., a communication network. Examples of communication networks include a local area network (“LAN”) and a wide area network (“WAN”), e.g., the Internet.

A number of embodiments of the invention have been described. Nevertheless, it will be understood that various modifications may be made without departing from the spirit and scope of the invention. Accordingly, other embodiments are within the scope of the following claims.

What is claimed is:

1. A dialysis machine comprising:
one or more processing units configured to transmit control data;
a pump configured to pump medical fluid to and from a patient based at least in part on control data received from the one or more processing units; and
a wireless communications interface configured to receive data from a mobile electronic device using a wireless communications protocol,
wherein the one or more processing units are configured to:
process input received from the wireless communications interface, and
determine the control data based on the processed input.

2. The dialysis machine of claim 1, wherein the one or more processing units are configured to cause a transmission, using the wireless communications interface, of user interface data to the mobile electronic device that enables the mobile electronic device to function as a remote user interface for the dialysis machine.

3. The dialysis machine of claim 2, wherein the user interface data enables the mobile electronic device to display, on a hardware display of the mobile electronic device, one or more selectable elements that correspond to respective selectable elements on a user interface display of the dialysis machine.

4. The dialysis machine of claim 3, wherein the one or more selectable elements comprise a selectable element for stopping the pump.

5. The dialysis machine of claim 3, wherein the one or more selectable elements comprise a selectable element for muting an alarm of the dialysis machine.

6. The dialysis machine of claim 2, wherein the user interface data enables the mobile electronic device to display, on a hardware display of the mobile electronic device, one or more parameters representing a current state of the dialysis machine.

7. The dialysis machine of claim 1, wherein the data received from the mobile electronic device includes data indicative of one or more particular positions on a user interface display of the dialysis machine.

8. The dialysis machine of claim 7, wherein the one or more processing units are configured to cause a cursor or pointer to be displayed on the user interface display of the dialysis machine at the one or more particular positions in response to receiving the data indicative of the one or more particular positions.

9. The dialysis machine of claim 7, wherein the data received from the mobile electronic device includes data indicative of a selection of a selectable element located at a particular position of the one or more particular positions, and wherein the one or more processing units are configured to cause a selection of the selectable element in response to receiving the data indicative of a selection of a selectable element.

10. A computer readable medium storing computer executable instructions that, when executed by a hardware processor of a mobile electronic device, carry out operations comprising:
receiving, from a dialysis machine, data representing a current state of the dialysis machine;
displaying, on a hardware display of the mobile electronic device, a user interface that enables control of the dialysis machine, based on the data representing a current state of the dialysis machine;
receiving, at the user interface, user input representing a command to be executed by the dialysis machine; and
transmitting, using a wireless communication protocol, data representing the command to be executed by the dialysis machine.

11. The computer readable medium of claim 10, wherein the user input represents a command to de-activate an alarm of the dialysis machine.

12. The computer readable medium of claim 10, wherein the user input represents a command to stop a pump of the dialysis machine.

13. The computer readable medium of claim 10, wherein the user interface comprises one or more selectable elements that correspond to respective selectable elements on a user interface display of the dialysis machine.

14. The computer readable medium of claim 10, wherein the user interface comprises one or more parameters representing the current state of the dialysis machine.

15. The computer readable medium of claim 10, the operations comprising:
receiving, at the user interface, user input representing movement of a pointer or cursor location on the user interface; and
transmitting, using the wireless communication protocol, data representing the movement to be executed by the dialysis machine.

16. The computer readable medium of claim 10, the operations comprising one or more operations that facilitate pairing the mobile electronic device and the dialysis machine.

17. A mobile electronic device comprising:
a hardware processor;
a wireless communications interface configured to transmit data using a wireless communications protocol; and
a movement sensor,
wherein the hardware processor is configured to receive data from the movement sensor representing movement of the mobile electronic device and, based on the received data, use the wireless communications interface to transmit, to a dialysis machine, data usable by the dialysis machine to affect a position of a cursor on a user interface of the dialysis machine.

18. The mobile electronic device of claim 17, wherein the hardware processor is configured to facilitate pairing of the mobile electronic device and the dialysis machine such that wireless communications can occur using the wireless communications protocol.

19. The mobile electronic device of claim 17, wherein the movement sensor comprises at least one of a gyroscope or an accelerometer.

20. The mobile electronic device of claim 17, further comprising a hardware display coupled to the hardware processor, wherein the hardware processor is configured to receive data representing a user input made to the hardware display and to transmit, to the dialysis machine, data usable by the dialysis machine for selecting a selectable element displayed on the user interface of the dialysis machine.

21. A dialysis machine comprising:
one or more processing units configured to transmit control data;
a pump configured to pump medical fluid to and from a patient based at least in part on control data received from the one or more processing units; and
a wireless communications interface configured to receive data from a mobile electronic device using a wireless communications protocol, wherein the data received from the mobile electronic device includes data indicative of one or more particular positions on a user interface display of the dialysis machine, and
wherein the one or more processing units are configured to cause a cursor or pointer to be displayed on the user interface display of the dialysis machine at the one or more particular positions in response to receiving the data indicative of the one or more particular positions.

22. The dialysis machine of claim 21, wherein the data received from the mobile electronic device includes data indicative of a selection of a selectable element located at a particular position of the one or more particular positions, and wherein the one or more processing units are configured to cause a selection of the selectable element in response to receiving the data indicative of a selection of a selectable element.

23. A dialysis system comprising:
a first dialysis machine comprising:
one or more first processing units configured to transmit control data;
a first pump configured to pump medical fluid to and from a first patient based at least in part on first control data received from the first one or more processing units; and
a first wireless communications interface configured to receive data from a mobile electronic device using a wireless communications protocol,
wherein the first one or more processing units are configured to process input received from the first wireless communications interface and to determine the first control data based on the processed input;
a second dialysis machine comprising:
one or more second processing units configured to transmit control data;
a second pump configured to pump medical fluid to and from a second patient based at least in part on control data received from the one or more processing units; and
a second wireless communications interface configured to receive data from a mobile electronic device using a wireless communications protocol,
wherein the second one or more processing units are configured to process input received from the second wireless communications interface and to determine the second control data based on the processed input; and
the mobile electronic device comprising a hardware processor and a third wireless communications interface configured to transmit data to the first and second dialysis machines using the wireless communications protocol.

24. The dialysis system of claim 23, wherein at least one of the first and second dialysis machines comprises a hemodialysis machine.

25. The dialysis system of claim 23, wherein at least one of the first and second dialysis machines comprises a peritoneal dialysis machine.

26. The dialysis system of claim 23, wherein the mobile electronic device is configured to transmit data associated with a first user interface of the first dialysis machine and configured to transmit data associated with a second user interface of the second dialysis machine.
